(19) United States
(12) Patent Application Publication
(10) Pub. No.: US 2023/0404668 A1
(43) Pub. Date: Dec. 21, 2023

(54) APPARATUS FOR LASER ENDO-VASCULAR ABLATION

(71) Applicant: Foreveryoung Technology Corporation, Lincoln, MA (US)

(72) Inventors: Yunfei Cao, Lincoln, MA (US); Joel Melnick, Waltham, VT (US); Changnian Han, Lincoln, MA (US); Ann Xin, Newark, CA (US)

(73) Assignee: Foreveryoung Technology Corporation, Lincoln, MA (US)

(21) Appl. No.: 17/840,836

(22) Filed: Jun. 15, 2022

(51) Int. Cl. A61B 18/24; G02F 1/355; G02F 1/35

(52) U.S. Cl. CPC A61B 18/245; G02F 1/3551; A61B 2018/00577; G02F 1/3503; G02F 1/3507; G02F 1/354

(57) ABSTRACT

A light processing apparatus includes a first non-linear crystal disk for transmitting a first beam of photons having a first frequency to a second beam of photons having the first frequency and a second frequency oscillating in polarization directions orthogonal to each other, the second frequency being a half of the first frequency. Further included is a waveplate for transmitting the second beam of photons to a third beam of photons by rotating polarization directions of the second beam of photons such that the photons of the first frequency and of the second frequency oscillate in the same polarization directions. A second non-linear crystal disk is configured to transmit the third beam of photons to a fourth beam of photons of the first frequency, the second frequency and a third frequency, the third frequency being approximate a third of the first frequency.

### TECHNICAL FIELD

This application relates generally to medical laser ablation instrument, particularly to laser ablation instrument to be used for removal of blockage in the blood vessels.

### BACKGROUND

Laser has been seen to be used in ablation of blockage in the blood vessels. Laser beams at a specific frequency 1-40 Hz having a wavelength in the proximity of 355 nanometer (nm) are deemed to have been effective in removing blockage in the blood vessels while causing least harm to other part of the tissues in the blood vessels. However, harvesting and isolating laser beams with a wavelength of 355 nm have been lack of efficiency, demanding excessive powers and wearing optical part.

### SUMMARY

In accordance with a first aspect of the present disclosure, there is set forth a light processing apparatus that includes a first non-linear crystal disk configured to transmit a first beam of photons having a first frequency to a second beam of photons having the first frequency and photons having a second frequency, the second frequency being approximately an half of the first frequency, the photons having the first frequency and the photons having the second frequency oscillate in polarization directions orthogonal to each other. The light processing apparatus further includes a waveplate configured to transmit the second beam of photons to a third beam of photons by rotating polarization directions of the second beam of photons such that the photons of the first frequency and the photons of the second frequency oscillating in approximately the same polarization directions. In addition, a second non-linear crystal disk is configured to transmit the third beam of photons to a fourth beam of photons of the first frequency, photons of the second frequency and photons of a third frequency, the third frequency being approximate a third of the first frequency.

Further disclosed is a method of light processing that includes providing a first non-linear crystal disk for transmit a first beam of photons having a first frequency to a second beam of photons having the first frequency and photons having a second frequency, the second frequency being approximately a half of the first frequency, the photons having the first frequency and the photons having the second frequency oscillate in polarization directions orthogonal to each other; providing a waveplate for transmitting the second beam of photons to a third beam of photons by rotating polarization directions of the second beam of photons such that the photons of the first frequency and the photons of the second frequency oscillating in approximately the same polarization directions; and providing a second non-linear crystal disk for transmitting the third beam of photons to a fourth beam of photons of the first frequency, photons of the second frequency and photons of a third frequency, the third frequency being approximate a third of the first frequency.

Yet further disclosed is an ablation apparatus that includes the light processing apparatus producing a laser light at a wavelength of approximately 355 nm according to the above. The a laser tissue ablation apparatus further includes a beam shaper receiving the laser light and produces a round shaped beam profile with desired diameter at the acceptance side of the catheter. Also included by the laser tissue ablation apparatus are a laser signal draw configured to measure the frequency of the laser light for calibration; a lens configured to focus the shaped laser light to focused laser light; a catheter configured to be inserted into a destination inside patient's body; and a flexible optical fiber connecting the lens and the catheter, configured to transmit the focused and shaped laser beam and deliver the same to the destination inside the patient's body for ablation.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosed systems and methods and are not intended as limiting. For purposes of clarity, not every component may be labeled in every drawing. In the following description, various embodiments are described with reference to the following drawings.

FIG. 1 is a schematic view of a laser generating apparatus in accordance with the present disclosure.

FIG. 2 is schematic view of a part of the optical apparatus including a component of second harmonic crystal in accordance with the present disclosure.

FIG. 3 is schematic view of a part of the optical apparatus including a component of waveplate in accordance with the present disclosure.

FIG. 4 is a schematic view of a part of the optical apparatus including a component of third harmonic crystal in accordance with the present disclosure.

FIG. 5 is a schematic view of a laser ablation apparatus including the laser generating apparatus in accordance with the present disclosure.

### DETAILED DESCRIPTION

The laser generating apparatus and the laser ablation apparatus described following as examples, are intended to generate laser beams with wavelength in the proximity of 355 nm. It should be appreciated that the scope and spirit of this disclosure is not limited to these examples. The example of using or not using certain component do not necessarily affect the scope of present disclosure. The term of optical component, such as second harmonic generation, second harmonic, frequency doubling, and similarly for other components described in the following, can be interchangeably used, and do not affect the scope of the present disclosure.

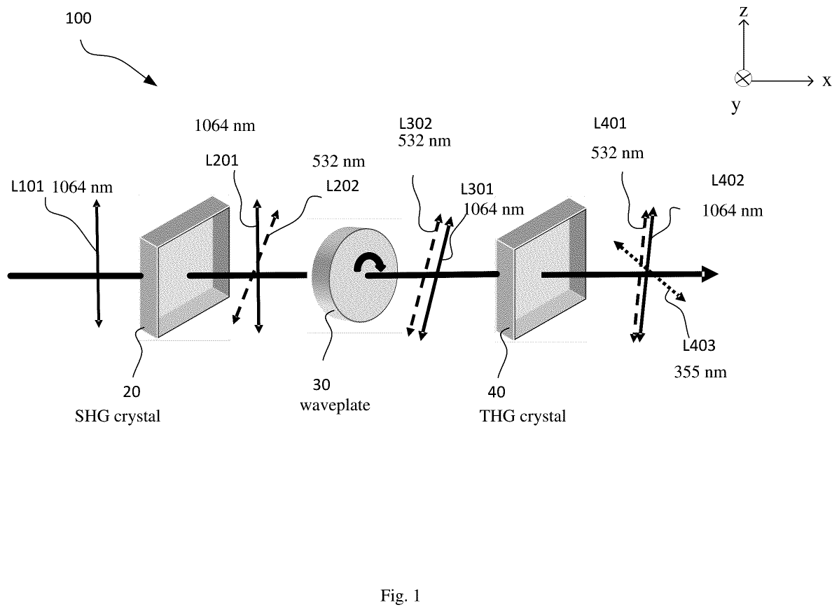

FIG. 1 is a schematic view of a laser generating apparatus in accordance with the present disclosure.

Referring to FIG. 1, laser generating apparatus 100 includes second harmonic generator (SHG) or second harmonic crystal 20, a waveplate 30 and a third harmonic generator (THG) or third harmonic crystal 40.

In an example embodiment, a beam of base laser L101 is used to produce the laser energy for performing ablation on blockage or partial blockage formed in blood vessels. Base laser L101 with a wavelength of 1064 nm enters into laser generating apparatus 100, traveling in direction X with its electric field oscillating in direction Z.

Base laser beam L101 enters into SHG 20 with a wavelength of 1064 nm, traveling in direction X and oscillating in direction Z and exits SHG 20 with a combined two laser components, L201 with a wavelength of 1064 nm oscillating in direction of Z and L 202 with a wavelength of 532 nm, oscillating orthogonally with L201 in direction of Y. As can be seen, L202 has doubled the frequency of L101.

As can be seen, only a portion of light L101 is transmitted to light L202 with double the frequency (a half of the wavelength, 532 nm). The rest of light L101 remains wavelength 1064 nm and the same oscillating direction as light L201.

Subsequently, light L201 enters waveplate 30 traveling in direction X and oscillating in direction Z. Light L202 enters waveplate 30 traveling in direction Y and oscillating in direction Z.

In this example embodiment, waveplate 30 is configured to rotate part of the light entering it, L201 to L301, from polarization direction in Z axis to polarization direction in Y axis, keeping the same wavelength 1164 nm and the same strength. As can be seen, waveplate 30 rotates light L201 without attenuating, deviating, or displacing the beam and it only effectuate the polarization rotation to one component of polarization (L201) with respect to its orthogonal component. Light component L202 in wavelength 532 nm is no affected, continues traveling in X direction, coming out from waveplate 30 with the same wavelength 532 nm, the same polarization direction in Y axis.

THG 40 which is a third harmonic generation or frequency tripling crystal, is configured to transmit base laser beams L301 and L302 with respective wavelengths of 1064 nm and 532 nm, both oscillating in direction Y to laser beam L403 of wavelength 355 nm, oscillating in Z axis, orthogonal to Y axis, and remnant laser beams of L402 and L401 with respective wavelengths of 1064 nm and 532 nm, oscillating in direction of Z.

It should be noted that the above descried parameters of light components can be in a range of a defined values. For example, the second frequency being approximately a half of the first frequency, with a range of 20%-60%. The third frequency being approximate a third of the first frequency with a range of 10%-50%.

Furthermore, the first beam of photons has a wavelength of approximately 1064 nm with a range of 1063 nm-1065 nm. The second beam of photons having the first frequency has a wavelength of approximately 1064 nm, with a range of 531 nm-533 nm, and photons having a second frequency has wavelength of approximately 532 nm. The laser beam L403 of approximately wavelength 355 nm with a range of 354 nm-356 nm.

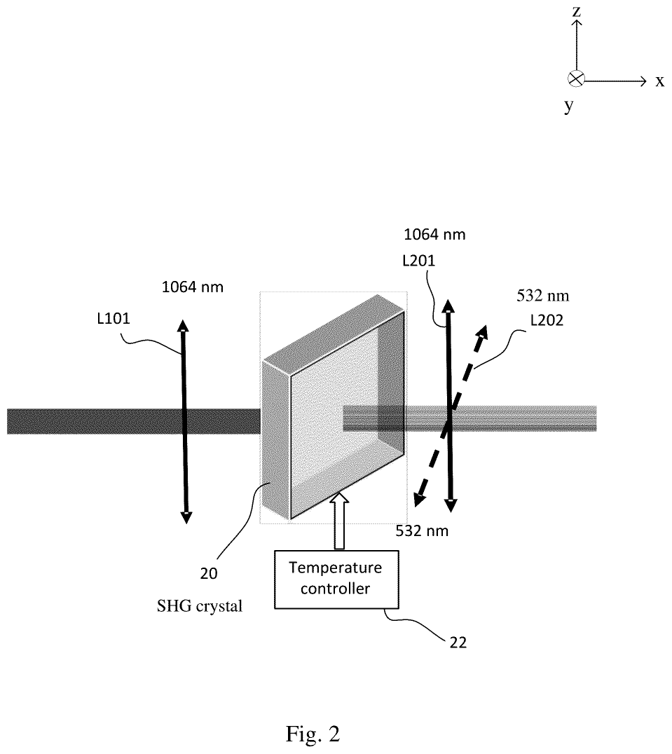

FIG. 2 is schematic view of a part of the optical apparatus including a component of second harmonic crystal in accordance with the present disclosure.

Referring to FIG. 2, SHG 20 is a second harmonic crystal. As known to those skilled in the art that second harmonic crystal in general provides frequency doubling or second harmonic generation which is a nonlinear optical process in which two photons with the same frequency interact with a nonlinear material, are “combined”, and generate a new photon with twice the energy of the initial photons (equivalently, twice the frequency and half the wavelength), that conserves the coherence of the excitation. It is a special case of sum-frequency generation (2 photons), and more generally of harmonic generation.

Base laser L101 with a wavelength of 1064 nm enters into SHG 20, traveling in direction X and oscillating in direction Z, and exits SHG 20 with laser beams of combined two laser components, L201 with a wavelength of 1064 nm oscillating in direction of Z and L202 with a wavelength of 532 nm, oscillating orthogonally with L201 in direction of Y. As such, SHG 20 is configured to transmit base laser beam L101 with a wavelength of 1064 nm oscillating in direction Z, to a combination of two laser components, L201 with a wavelength of 1064 nm oscillating in direction of Z and L202 with a wavelength of 532 nm, oscillating orthogonally with L201 in direction of Y. As can be seen, light L202 has a frequency double of that of light L101. That is that SHG 20 is configured to double the frequency and change the oscillating direction of part of light L101, turning it into light L202.

As can be seen, only a portion of light L101 is converted to light L202 with double the frequency (a half of the wavelength, 532 nm). The rest of light L101 remains wavelength 1064 nm and the same oscillating direction as light L201. The converting efficiency can be expected to be between 30% to 70%.

Barium borate (BBO) is one kind of nonlinear crystals that's known to those skilled in the art to have large nonlinear coefficients, high threshold for laser damage, and low thermo-optic coefficient. It is made in a way to be suitable for use in harmonic generation operations, optical parametric oscillators.

To maximize the conversion efficiency of SHG, phase velocity of the SHG 20 and incoming fundamental wave L101 need to be matched. Such condition is known as phase-matching and is realized by selecting the angle of the optic axis with regard to the laser propagation direction, known as cutting angle. In one embodiment, SHG 20 is constructed by using BBO crystal in type I phase-matching condition, with the cutting angles in a range of 20 to 27 degrees. Yet in another embodiment, BBO crystal is constructed with cutting angle of 23 degrees. The cutting angle θ of BBO crystal that determines the phase-matching condition is calculated according to,

n_{o,1064,T}(θ)=n_{e,532,T}(θ) Eq. (1)

wherein, θ is the cutting angle, n_{o,1064,T} is the refractive index for ordinary wave with wavelength of 1064 nm at the temperature T set by temperature controller, and n_{e,532,T} is the refractive index of extraordinary wave of wavelength 532 nm at the setting temperature T. The cutting angle θ is known once the temperature T is chosen.

Referring to FIG. 2, temperature controller 22 is configured to control the temperature within SHG crystal 20. As known to those skilled in the art, conversion efficiency (between 10% to 70%) of SHG crystal is affected by its temperature. In this example embodiment, temperature is preferable control according to the following equation:

n_{o,1064,T}(θ)=n_{e,532,T}(θ)

As such this configuration of temperature serves the purpose of maintaining stable conversion efficiency regardless of ambient temperature. In most cases, the temperature T is set higher than the ambient temperature for simplicity of the control system.

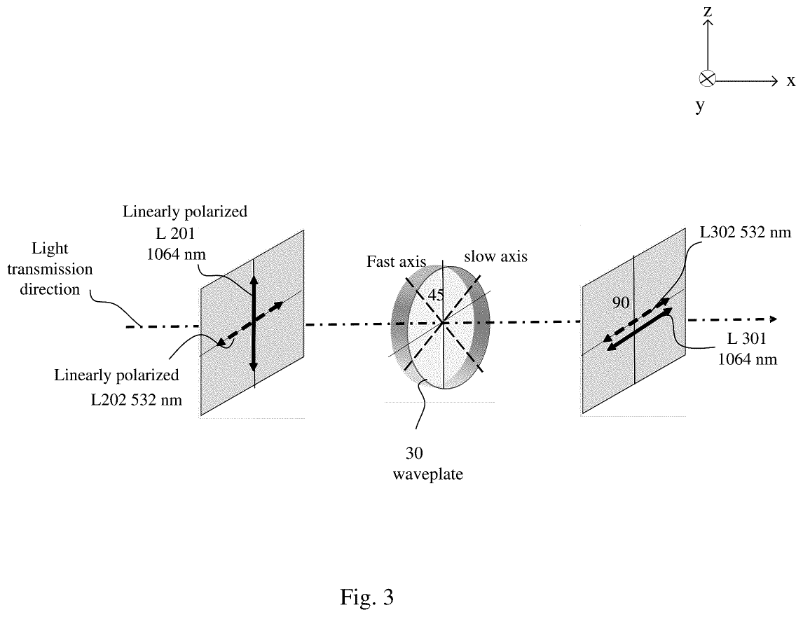

FIG. 3 is schematic view of a part of the optical apparatus including a component of waveplate in accordance with the present disclosure.

Waveplate 30 is a kind of crystal that performs an optical operation referred as “phase matching”. As known to those skilled in the art, waveplates, also known as retarders, transmit light and modify one component of the polarization state without attenuating, deviating, or displacing the beam. Waveplates achieve this by retarding (or delaying) one component of polarization with respect to its orthogonal component. There are multiple ways of application that waveplate can be used for. One example is to alter the existing polarization of an optical energy. For example, lasers are typically horizontally polarized. If it is needed for laser light to reflect off a metallic surface, then this can be a problem because mirrors work best with vertically polarized light. In this example embodiment, to optimize the reflectivity of the metallic surface, a λ/2 (a half wavelength) waveplate with its axes oriented preferably at 45° can be used to rotate a horizontally polarized laser to vertical.

In the example embodiment shown in FIG. 3, when it is desirable to adjust the polarization axis to any other orientation. Rotating the waveplate axis an angle of θ from the incident polarization will rotate the exiting polarization by 2θ. Since waveplates are highly parallel, inserting or rotating a λ/2 waveplate can reconfigure an entire optical setup with no realignment.

As shown in FIG. 3, waveplate 30 is constructed by using a dual wavelength waveplate to achieve the rotation of the polarization angle of laser 1064 nm laser while keeping the polarization of 532 nm laser not changed.

In this example embodiment, in order to rotate the polarization angle of laser 1064 nm laser 90° to be aligned with the polarization angle of 532 nm laser, waveplate 30 is configured to have the rotation mount of a fast axis of the waveplate to be at 45° to the polarization of the laser 1064 nm laser.

Further in this example embodiment, in order to maintain the polarization angle of 532 nm laser unchanged while rotating the polarization angle of laser 1064 nm laser 90°, the waveplate is constructed with Calcite or Quartz crystal. And the thickness d of this crystal is calculated according to,

$$\{ \begin{matrix}d \cdot ( {n}_{e , 1064} - {n}_{o , 1064} ) = 1.064 µm \times ( {m}_{1} + 1 / 2 ) \\ d \cdot ( {n}_{e , 532} - {n}_{o , 1064} ) = 0.532 µm \times {m}_{2}\end{matrix} \qquad Eq . ( 2 )$$

wherein,
d is thickness of an integral waveplate with multiple order waveplates or the difference between two multiple order waveplate if two combined waveplates with optical axis rotated 90 degrees to each other are used;
m₁ and m₂ are integer numbers, n is the refractive index.

Since the polarization of the 1064 nm and 532 nm might not be linear after passing a waveplate, fitting the thickness of the waveplate according to Eq. 2 helps achieve to keep the rotation of the direction of 1064, leaving 532 nm unchanged while keep both laser linearly polarized, and therefore achieving the alignment of the polarization of the 1064 nm and 532 nm.

Waveplate 30 may be constructed of a multiple order waveplates or a combination of two multiple order waveplates.

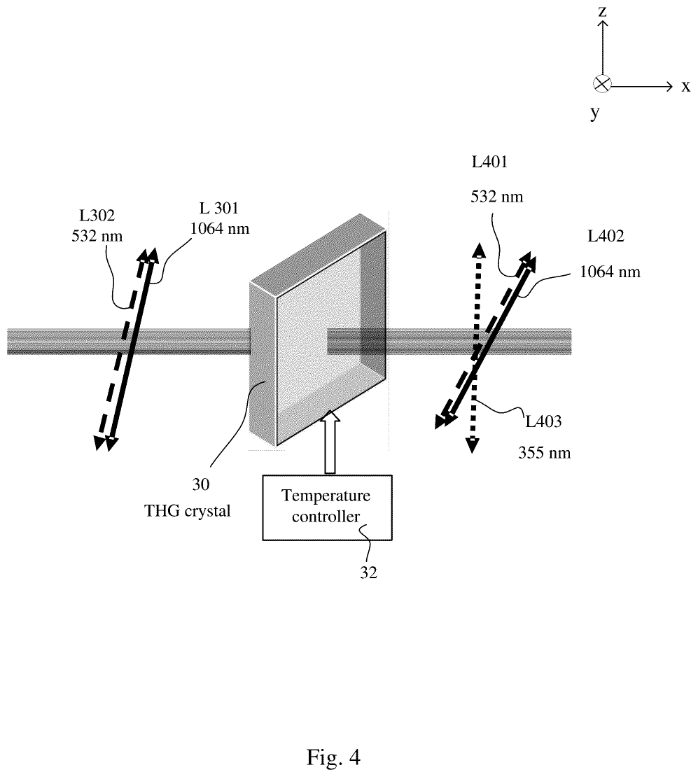

FIG. 4 is a schematic view of a part of the optical apparatus including a component of third harmonic crystal in accordance with the present disclosure.

In a THG (third harmonic generation) setup, the non-linear crystal produces a “frequency tripling” phenomenon in which an input light beam is converted to an exiting light beam with three times the optical frequency of the input light beam. In the process, three photons from base laser are converted into a single photon at three times the light frequency of the base laser (one-third the wavelength). In principle, that can be achieved with a χ(3) nonlinearity for direct third-harmonic generation, but this is difficult due to the small χ(3) nonlinearity of optical media and also because of phase-matching constraints (except for tripling in gases). Therefore, frequency tripling is usually achieved as a cascaded process, beginning with frequency doubling of the input beam and subsequent sum frequency generation of both waves, with both processes being based on nonlinear crystal materials with a χ(2) nonlinearity.

The direction of the polarization in the example embodiment is of a difference by 20 degrees, depending on the final selection of the crystal cutting angle.

A common approach is to use two BBO (Beta Barium Borate) crystals, or an LBO crystal and a BBO crystal, the first being phase-matched for second-harmonic generation and the second for sum frequency generation. It is easy to make this process efficient when using pulses from a Q-switched or mode-locked laser, but also possible in continuous-wave operation, e.g. with intracavity frequency doubling and resonant sum frequency generation.

Temperature controller 32 is configured to control the temperature within THG crystal 30. As known to those skilled in the art, conversion efficiency (what attributes) of THG crystal is affected by its temperature. In this example embodiment, temperature is preferable control according to the following equation:

n_{o,1064,T}+n_{o,532,T}=2×n_{e,355,T} Eq. (3)

As such this configuration of temperature serves the purpose of maintaining stable conversion efficiency regardless of the ambient temperature.

One of the novel aspects of the embodiments shown in the present disclosure is that the light elements, L201 and L202 are phased matched efficiently by waveplate 30. Phased matched light components, L301 with wavelength 1064 nm and L302 with wavelength 532 nm oscillating in the same direction, resulting higher energy when combined (frequency summing). As a result, the conversion energy efficiency (10%-70%) is optimized.

The following deduction explains how, in this example embodiment, energy efficiency is improved by phase matching of sum frequency generation in simplified plane wave condition.

$${I}_{3} = {{I}_{3}^{( max )} [ \frac{sin ( \frac{Δ k L}{2} )}{( \frac{Δ k L}{2} )} ]}^{2} ; \qquad Eq . ( 4 )$$

and the energy efficiency C is expressed as:

C=I₃/I₁; Eq. (5)

wherein I₃ is the intensity of the generated sum frequency light, I₃^{(max)} is the maximum achievable laser intensity of the sum frequency wave L403, and I₁ is the intensity of the fundamental wave. Δk=k₁+k₂−k₃, and is the wave factor mismatch of the three lights involved in the process, wherein k₁, k₂, and k₃ are the wave factor inside the crystal of the fundamental wave L101, SHG wave L202, and the THG wave L403, respectively.

To convert the sum-frequency light wave efficiently, the mismatch factor AU, must be small, because the second part of the equation

$${[ \frac{sin ( \frac{Δ k L}{2} )}{( \frac{Δ k L}{2} )} ]}^{2}$$

is monopoly decreases with increased Δk when the crystal length L is selected. For instance, ΔkL=1 can cause −8% of loss on efficiency, while ΔkL=2 can cause −29% of loss on efficiency.

Theoretically, the total power conversion efficiency of the frequency tripling process could be close to 100% in a single pass through the crystals. For that, the frequency doubler should have a conversion efficiency of 2/3, so that the second-harmonic wave has twice the power of the remaining fundamental wave, and both have equal photon numbers. In practice, the efficiency of the frequency doubler is normally somewhat lower (often around 40 to 50%), and in particular the sum frequency mixer is far from 100% efficient. The latter problem can result from many effects, such as too low optical intensities, design limitations enforced by optical damage, effects of spatial walk-off, mismatch of pulse duration and/or temporal walk-off, etc. Tentatively, the conversion works best for high peak powers in not too short (e.g. picosecond) pulses, and when the beam quality is high and the optical bandwidth not too high. Overall conversion efficiencies from infrared to ultraviolet can then be of the order of 30 to 40%.

To improve the conversion efficiency of the THG wave L403, all remain fundamental wave L201 and SHG L202 are expected to contribute to the sum-frequency process in the THG crystal 40 with paralleled polarization. Hence, the rotation of the dual wavelength waveplate controls the polarization angle of the 1064 nm while the polarization angle of the 532 nm remains.

As such, this configuration enables one of the novel aspects of the present disclosure which allows for achieving the laser of 355 nm with high energy conversion efficiency.

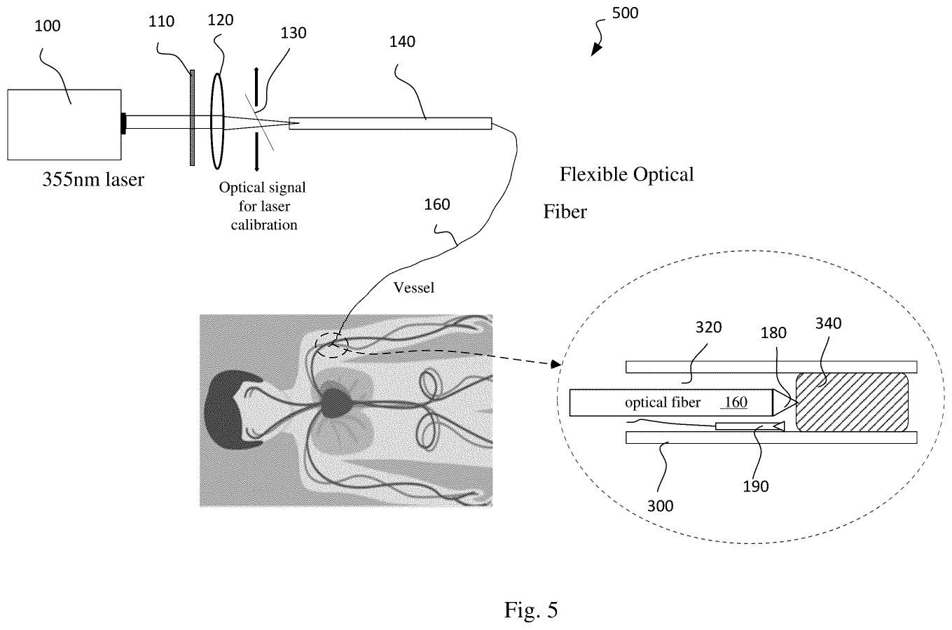

FIG. 5 is a schematic view of a laser ablation apparatus 500 including the laser generating apparatus in accordance with the present disclosure.

Referring to FIG. 5, in one example embodiment, laser ablation apparatus 500 includes laser generating apparatus 100, a beam shaper 110, a laser signal draw 130, a lens 120, a catheter 140, a flexible optical fiber 160, a laser application head 180, and a blood vessel monitor 190.

Laser generating apparatus 100 as described above is for generating laser beams of a desirable wavelength, 355 nm in this embodiment. Beam shaper 110 is configured to shape the cross-section contour of the laser beams in any desirable shape and can adopt the design of any laser beam shapers. Laser signal draw 130 is an optical conduit, such as a fiber conduit, used to draw optical signal for laser calibration. Laser calibration includes adjusting temperatures of one or both of temperature controllers 22 and 32. Along with beam shaper 110, focusing lens 120, such as a plano-convex lens, is configured to create a beam profile that delivers light efficiently into catheter while keep the catheter from being damaged by the intense light. Catheter 140 is a conduit assisting the insertion of flexible optical fiber 160 into a patient's blood vessel. Laser application head 180 is configured to allow optical fiber 160 to be inserted into the blood vessel 300, focus and apply laser energy onto blockage 340. Flexible optical fiber 160 is configured to transmit the laser energy from laser generating apparatus 100 to blood vessels monitor 190 is an optical receiver that is configured to receive optical signals indicating the situation of the blockage. It can be in a form of, for example, a catheter in combination of a video bogie, etc. Blood vessels monitor 190 is bundled with optical fiber 160 to be inserted into or pulled out together of the blood vessels.

Additionally, it is contemplated that systems, devices, methods, and processes of the present application encompass variations and adaptations developed using information from the embodiments described in the following description. Adaptation or modification of the methods and processes described in this specification may be performed by those of ordinary skill in the relevant art.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

1. A light processing apparatus, comprising:
a first non-linear crystal disk configured to transmit a first beam of photons having a first frequency to a second beam of photons having the first frequency and photons having a second frequency, the second frequency being approximately a half of the first frequency, the photons having the first frequency and the photons having the second frequency oscillate in polarization directions orthogonal to each other;
a waveplate configured to transmit the second beam of photons to a third beam of photons by rotating polarization directions of the second beam of photons such that the photons of the first frequency and the photons of the second frequency oscillating in approximately the same polarization directions; and
a second non-linear crystal disk configured to transmit the third beam of photons to a fourth beam of photons of the first frequency, photons of the second frequency and photons of a third frequency, the third frequency being approximate a third of the first frequency.

2. The light processing assembly of claim 1, wherein,
the first non-linear crystal disk is a second harmonic crystal disk.

3. The light processing assembly of claim 1, wherein,
the second non-linear crystal disk is a third harmonic crystal disk.

4. The light processing assembly of claim 1, wherein the first beam of photons has a wavelength of approximately 1064 nm, the second beam of photons having the first frequency has a wavelength of approximately 1064 nm, and photons having a second frequency has wavelength of approximately 532 nm.

5. The light processing assembly of claim 4, wherein,
the waveplate is configured to maintain the polarization direction of the photons of wavelength of 1064 nm while rotate the polarization direction of the photons of wavelength of 532 nm orthogonally.

6. The light processing assembly of claim 1 further comprising a first temperature controller to control a first temperature of the first non-linear crystal disk to adjust the temperature.

7. The light processing assembly of claim 1 further comprising a second temperature controller to control a second temperature of the second non-linear crystal disk to adjust the temperature.

8. The light processing assembly of claim 1 further comprising a ratable mount to control the orientation of the waveplate.

9. A method of light processing, comprising:
providing a first non-linear crystal disk for transmit a first beam of photons having a first frequency to a second beam of photons having the first frequency and photons having a second frequency, the second frequency being approximately an half of the first frequency, the photons having the first frequency and the photons having the second frequency oscillate in polarization directions orthogonal to each other;
providing a waveplate for transmitting the second beam of photons to a third beam of photons by rotating polarization directions of the second beam of photons such that the photons of the first frequency and the photons of the second frequency oscillating in approximately the same polarization directions; and
providing a second non-linear crystal disk for transmitting the third beam of photons to a fourth beam of photons of the first frequency, photons of the second frequency and photons of a third frequency, the third frequency being approximate a third of the first frequency.

10. The method of light processing of claim 9, comprising:
the first non-linear crystal disk is a second harmonic crystal disk.

11. The method of light processing of claim 9, wherein,
the second non-linear crystal disk is a third harmonic crystal disk.

12. The method of light processing of claim 9, wherein the first beam of photons has a wavelength of approximately 1064 nm, the second beam of photons having the first frequency has a wavelength of approximately 1064 nm, and photons having a second frequency has wavelength of approximately 532 nm.

13. A laser tissue ablation apparatus, comprising:
a light processing apparatus producing a laser light at a wavelength of approximately 355 nm;
a beam shaper receiving the laser light and produces a round shaped beam profile with desired diameter at the acceptance side of the catheter;
a laser signal draw configured to measure the frequency of the laser light for calibration;
a lens configured to focus the shaped laser light to focused laser light;
a catheter configured to be inserted into a destination inside patient's body; and
a flexible optical fiber connecting the lens and the catheter, configured to transmit the focused and shaped laser beam and deliver the same to the destination inside the patient's body for ablation.

14. The laser tissue ablation apparatus of claim 13, wherein the light processing apparatus comprising:
a first non-linear crystal disk configured to transmit a first beam of photons having a first frequency to a second beam of photons having the first frequency and photons having a second frequency, the second frequency being approximately an half of the first frequency, the photons having the first frequency and the photons having the second frequency oscillate in polarization directions orthogonal to each other;
a waveplate configured to transmit the second beam of photons to a third beam of photons by rotating polarization directions of the second beam of photons such that the photons of the first frequency and the photons of the second frequency oscillating in approximately the same polarization directions; and
a second non-linear crystal disk configured to transmit the third beam of photons to a fourth beam of photons of the first frequency, photons of the second frequency and photons of a third frequency, the third frequency being approximate a third of the first frequency.

15. The laser tissue ablation apparatus of claim 13, wherein,
the first non-linear crystal disk is a second harmonic crystal disk.

16. The laser tissue ablation apparatus of claim 13, wherein,
the second non-linear crystal disk is a third harmonic crystal disk.

17. The laser tissue ablation apparatus of claim 13, wherein the first beam of photons has a wavelength of approximately 1064 nm, the second beam of photons having the first frequency has a wavelength of approximately 1064 nm, and photons having a second frequency has wavelength of approximately 532 nm.

18. The laser tissue ablation apparatus of claim 13, wherein,
the waveplate is configured to maintain the polarization direction of the photons of wavelength of 1064 nm while rotate the polarization direction of the photons of wavelength of 532 nm orthogonally.

19. The laser tissue ablation apparatus of claim 13 further comprising a first temperature controller to control a first temperature of the first non-linear crystal disk to adjust the temperature.

20. The laser tissue ablation apparatus of claim 13 further comprising a second temperature controller to control a second temperature of the second non-linear crystal disk to adjust the temperature.
